(19) United States
(12) Patent Application Publication
(10) Pub. No.: US 2016/0287332 A1
(43) Pub. Date: Oct. 6, 2016

(54) Automated Surgical Instruments and Processes

(71) Applicant: Robert Kenneth Griffits, Coal Point (AU)

(72) Inventor: Robert Kenneth Griffits, Coal Point (AU)

(21) Appl. No.: 15/037,372

(22) PCT Filed: Nov. 20, 2014
(86) PCT No.: PCT/AU2014/050365

§ 371 Date: May 18, 2016

(30) Foreign Application Priority Data
Nov. 20, 2013 (AU) 2013904478

(51) Int. Cl. A61B 18/20; A61F 9/008; A61B 17/3203

(52) U.S. Cl. CPC A61B 18/20; A61B 17/3203; A61F 9/00825; A61B 18/201; A61B 2018/00577

(57) ABSTRACT

An Automated Surgical instrument is disclosed that is computer controlled and provided with a high degree of autonomy for performing automated procedures within humans and other animals. It may be mobile under its own control and may include a plurality of means to disrupt tissue including lasers and water jets. A method of providing barriers to prevent unwanted damage to tissue is also described. The instrument may be used to construct both artificial and biological structures in-vivo by taking advantage of 3D printing techniques made available by the flexible laser system disclosed, a selection of micro tools, and raw material delivery to the worksite for printing to the target area. The use of vibration generated electricity may avoid the need for wires or batteries. A particular embodiment for automated cataract surgery is described.

### BACKGROUND TO CATARACT SURGERY

A Cataract is the loss of clarity in the Crystalline Lens of an Eye.

Operations to remove the cloudy “Cataractous Lens” and help restore at least some degree of visual clarity have been around since Antiquity, with most of the advances occurring during the past 100 years.

Following World War II, Intraocular lenses, made of a variety of polymers, started to be utilised to replace the optical component that was previously rendered by an animal Crystalline Lens.

The technique of Cataract removal has proceeded through a pathway of evolution, with the occasional giant steps of technological revolution.

Early to Mid 20^{th} Century Cataract Technique involved dividing (either by Physical or Chemical means) the supporting structures (the Lens Zonules), which hold the Crystalline lens in situ, and expelling, the entire lens structure out through a large wound. This Technique was called “INTRACAPSULAR LENS EXTRACTION”.

By the middle part of the latter stage of the 20^{th} Century the technique had evolve to a technique known as “EXTRACAPSULAR LENS EXTRACTION”. This technique involved leaving the Lens Capsule in place, and removing the contents of the lens (comprising the Nucleus, and Cortex material), through a moderate sized opening of the front surface of the Lens Capsule, and through a moderately large (7 to 10 mm) external wound.

A key goal of the surgery is to maintain the integrity of the lens Capsule within the minimal limits that are necessarily imposed by the need to remove the Intracapsular Contents, and to place a Polymer Artificial Optical Lens within the Capsular Structure.

Placement of the Artificial Lens within the Capsular Structure, or alternatively in front of the Capsular Structure allows the Artificial Lens to be adequately positioned and supported in a stable location.

The early EXTRACAPSULAR LENS EXRACTION technique involved expelling the harder nuclear material out of the Capsular Structure, and out of the eye through a moderate sized wound of the external eye wall (namely either the Cornea, Sclera, or the junction of the two structures called the Limbus)

By the late 1980's, a new technique known as “PHACOEMULSIFICATION” was developed and became available. This allowed the micro-fragmentation of the harder nuclear material, which could be removed, either from within the capsular structure, or from just anterior to that structure. This was a significant advance, as a much smaller external eye wound could be utilised.

Techniques for cataract treatment have been advanced by phacoemulsification surgery, which permitted the fragmentation and removal of the lens material by high frequency ultrasound and suction with irrigation. The Capsule Structure and its supporting Zonular apparatus are left largely intact, except for the anterior capsulotomy (the incision in the capsule through which the cataractous lens is removed and its replacement inserted). Preservation of this inert ocular structure (i.e. capsule and zonular apparatus) supplies a mechanism by which intraocular lenses can be implanted so that they do not impinge on vital structures (as occurs with angle or iris supported intraocular lenses) and thus avoids chronic complications such as uveitis, glaucoma and corneal decompensation.

During phacoemulsification surgery the anterior crystalline lens capsule is torn away to form a circular opening by which the lens material can be removed (continuous curvilinear capsulorhexis). This produces a strong capsular rim that resists tearing even when stretched—generally improving the safety margin during surgery.

In extra-capsular surgery, a so-called can-opener capsulotomy is performed, but the irregular edges of the capsule are prone to radial capsular extension tears which can result in loss of vitreous or the lens into the vitreous, both adverse events which frequently result in a sub-optimal result and complications.

The external eye wound was by now largely dictated by the opening required to insert the Artificial Intraocular Lens. The later advent of foldable Intraocular Lense allowed further reduction in wound size.

In the first decade of the 21^{st} Century, a new Technique known as “FEMTOSECOND LASER ASSISTED CATARACT SURGERY” was developed. This technique utilised a Femtosecond Laser to create the external wounds, create a round opening in the front surface of the Lens Capsule, and partially fragment the Nucleus Material.

One objective of the present invention applying the described Automated Surgical Probe aims to further the evolution of Cataract Surgery by performing the Surgery within the Capsular Structure, rather than within the combined volume of the Lens Capsule and Anterior Chamber, thereby further reducing the Anatomical Disruption of the Eye. This may also provide additional protection to the Corneal Endothelial surface, which presently sustains at least some degree of trauma with all current Cataract Surgery techniques. It is also anticipated that an application of the present invention may further expand the scope and safety of Femtosecond laser assisted cataract surgery.

### SUMMARY OF THE INVENTION

The present invention, whilst not limited to, has particular application to computer controlled and or computer facilitated in-vivo animal (including human) intervention using suitably constructed instruments (that unless otherwise specifically identified may be generically referenced as Automated Surgical Probes or ASP's) for insertion into one or more in-vivo tissues and or cavities (preferably natural and or synthesised).

In this specification the use of examples is not necessarily intended to limit other embodiments and provision of an example(s) is not intended to imply that any one or more of said example is necessarily required: either absolutely or in particular embodiments. The term ‘and or’ preferably indicates that any one or more of the items joined by ‘and or’ may be applicable. Plural items linked by commas or ‘or’, are preferably understood as: any one or more of the items may apply, unless the context suggests a selection of one (or more) from the larger plurality. The use of ‘and’ preferably indicates (where the context is appropriate) that all items linked by ‘and’ are required unless a subset is nominated (eg any one of the claims selected from 1, 2 and 3.

Medicine is set to benefit significantly from the convergence of a number of advanced technologies, including a) the ultra-miniaturisation of electronic circuits, b) nano motors and machines (eg MEMS—Micro Electric Mechanical Systems), c) highly integrated micro/nano mirrors/lenses operable by said nano motors, d) biotechnology, e) nucleic acid sequencing, f) on chip path labs and cell substrate synthesis, g) integration of electronics with living tissue, h) carbon nanotubes, i) 3d printing and j) the increasing ability to target drugs, dyes, antibodies, proteins, markers, etc to specific cell types and or intracellular components. Delivering one or more of the preceding non-limiting examples to their target tissue/cells and controlling/co-ordinating them is likely to be a challenge that one or more disclosures in this specification may facilitate. It is further anticipated that the ASP's themselves may evolve into increasingly reduced packages. One end of the scale may target macro tissue structures (eg the lens of an eye, bone marrow, bowel polyps, neoplastic tissue) with the other end of the scale targeting individual cells with the ultimate goal to enter a cell and target specific structures within the cell for deletion/modification/enhancement, preferably using hybrid structures of nano/pico electronics and organic nano/molecular structures. It is anticipated that larger ASP's may be used for delivery of smaller ASP's.

It is preferable that an ASP may be inserted into the animal and remain in place for a period of time enclosed by animal tissue while continuing to function. It is also preferable that an ASP may be inserted into the animal and remain in place for a period of time enclosed by animal tissue with one at least tubes/wires leading externally and continue to function.

In this specification the term electromagnetic frequencies preferably include one at least of infra-red, visible and ultraviolet as non-limiting examples. The source of said electromagnetic frequency preferably including laser light and or other high intensity light source.
Non-limiting examples of said laser preferably include one at least of:
   external to the animal and directed through a transparent tissue—for example directed via: the anterior chamber of the eye to the lens region, or an orifice or an incision.
   a laser external to the eye with its output transmitted via a fibre-optic cable coupled to an ASP
   A laser(s) that is(are) located internal to the animal (eg part of and or coupled to an ASP (for example, one at least semiconductor laser diodes (see Photonic Energy Device or PED later in this disclosure).
An ASP preferably may include and or be coupled to a plurality of different lasers and or laser frequencies.
   It is further preferable that two at least lasers may be separately directed at a target wherein each has sub critical output (eg insufficient to cut/ablate, or insufficient to transform a dye, melt plastic in 3d printing) that becomes critical when they combine in phase (for example) at said target. Non-limiting examples applications of this preferably allow for one at least of:
      gang a plurality of low cost semiconductor laser diodes into a cheap/disposable precision effective laser,
      allowing laser frequencies to be used that are not readily available otherwise (for example, it is difficult to send femtolaser light down a fibre-optic channel, however by combining plural sources, this problem may be overcome.
      allow precise targeting in 3 dimensions for 3d shape cutting, tissue protection, 3d printing,
      accurately mark boundaries in 3d,
      by varying the coherence of the two (or more) beams, fine tuning of tissue damage or the type of tissue, cells or intracellular affected by laser energy.
Non-limiting examples of the use of said lasers preferably may include one at least of:
   tissue disruption, tissue welding (tissue to tissue, or tissue to synthetic), identification marking (eg of boundaries), fluorescing, flipping biphasic material state, 3d printing, deletion/modification of synthetic structures.

Although the specification usually refers to laser as the source of electromagnetic radiation for use with the invention, it is preferable that other sources are permitted where applicable. For example, non-coherent ultraviolet or visible light may be of use in setting or destroying particular polymers. Non-limiting examples of said non-coherent light sources preferably may include one at least of: LEDs and OLEDS

Non-limiting examples of said in-vivo intervention preferably may includes one at least of:
a) ‘tissue disruption’: that unless otherwise further clarified in this document preferably includes one at least of: cutting, disruption, destruction, transformation, ablation, interference with, removal and or similar processes to one or more tissues or parts thereof within said living animal.
   Non-limiting examples of said tissue disruption preferably may include:
      i) part at least of the lens substrate from within the lens capsule of an eye;
      ii) one at least constituents of bone marrow in part at least,
      iii) malignant tissue
      iv) damaged tissue,
      v) pathogens
      vi) healthy tissue.
   It is preferable that said disruption may vary from a macroscopic attack on tissue (eg fragmentation of the lens of an eye) to targeting on a cellular (eg neoplastic cells) or sub-cellular (eg aberrant DNA, RNA, Proteins) level.
   Non-limiting example approaches to tissue disruption preferably may include one at least of:
      ultrasound (eg phacoemulsification in cataract surgery),
      laser,
      fluid jet (eg water jet),
      cautery,
      physical disruption (eg pushing an ASP through tissue, cutting (eg micro pincers/shears, spinning blade)),
      targeted delivery of immune system components (eg cells, antibodies)
      dissolution using organic and or inorganic substances,
      focused delivery of cytotoxic drugs,
      local variation of pH,
      local variations to tissue oxygenation.
b) ‘Barrier’: insertion, enhancement, construction, removal, modification, transformation and or state change, in part at least within said living animal.
   Non-limiting examples of said barrier preferably may include:
      Barrier to protect tissues/regions from the effect of one at least electromagnetic frequencies used for processes described for an ASP, the barrier preventing or reducing the transmission of said electromagnetic frequencies (eg, opaque to infra-red light), wherein, for example, the barrier is for insertion: into a natural cavity and or a fabricated cavity/void; non-limiting examples preferably including one at least of:
         flowable (eg water soluble or elasto-viscous) into a natural and or fabricated cavity/void, non-limiting examples preferably including one at least of:
         india ink
         mix of starch and iodine
         biocompatible infra-red absorbing dyes
         suspension of plastic micro/nano beads (said plastic preferably biodegradable),
         photobiphasic material—increased opacity with 1^{st} frequency of electromagnetic radiation (EMR) and reduced opacity with 2^{nd} frequency of EMR,
         carbon nanotubes and or nanoelectronics—preferably self assembling and provided in suspension.
         Fabricated protective barrier—eg 3 D printing.
      Barrier to indicate a particular area/region, preferably to computer aided detection apparatus. For example:
         a dye that fluoresces upon exposure to particular frequency(s) of EMR.
         For example: a dye that fluoresces when hit by a laser may be detected by a digital imaging system to indicate that a ‘No Go Zone’ has been reached.
         a coloured fluid placed into a particular space (eg between lens and lens capsule of the eye) wherein a computer coupled to digital imaging may recognise (or dynamically learn to recognise) a particular shape and be able to determine if this shape changes (eg image of coloured fluid in lens space may indicate that the periphery of the lens has been reached).
         an indicating barrier may include a plurality of micro/nano electronic devices (eg they preferably may be suspended in fluid that is infused into a space or fixed to adjacent structures)—said devices preferably may monitor one at least of:
         One at least EMR frequency (eg UN, light, infra-red), Pressure, Temperature, Vibration, Chemicals (eg cytotoxic), pO2, pH; as non-limiting examples.
         and report back status (preferably dynamically) with said reporting preferably wireless. Said devices preferably may be powered by one at least of vibration and or power derived from surrounding nutrients (eg glucose/oxygen) as non-limiting examples.
      Barrier to segregate different components, preferably with selective permeability. For example in a patient with a haemopoeitic neoplasm, chambers may be constructed and healthy cells selectively sorted to a particular compartment and neoplastic cells to another.
      Packaging Barrier—for example a Bowel ASP may remove a polyp, package it in a membrane and attach it to the ASP for subsequent removal with the ASP.
   It is preferable that one or more barriers may be removed in part at least. For example, suction of fluid barriers, destruction by laser, fluid jet or ultrasound, biodegradable, conversion to more readily eliminated format (or useable for constructing other materials).
c) Construction, preferably using synthetic and or biological materials.
d) Delivery of materials to an ASP and or tissue. Non-limiting examples of said delivery preferably may include one at least of:
   Delivery from external to the animal—eg using pipes/tubes/conduits.
   Fabrication of materials from external deliveries and or available in-vivo resources.
e) Removal of materials. Non-limiting examples of said removal preferably may include one at least of:
   Removal to the outside (or elsewhere in the body) by using pipes/tubes/conduits, Biodegradation, Destruction, Conversion, Consumption.
f) maintaining a safe in-vivo environment.

It is preferable that ASP's may include one at least of the following non-limiting attributes:
may be for insertion and subsequent removal.
physical connection (eg tubes, power, fibre-optic) to external apparatus.
autonomous of external physical connection.
to remain indwelling—said indwelling preferably may include ASP's fabricated to biodegrade and or self degrade in-vivo, in part at least
to be modifiable in-vivo: eg self construct additions, alterations and or reductions, preferably using a combination of biological and non-biological substrates.

The invention preferably allows for operator intervention/assistance with one or more processes.

It is preferable that said computer (for example, one at least of: digital computer, analogue computer, neural network, DSP, microprocessor, and or hardware arranged for executing a computer program) control includes one at least of:
a) one at least computers external to the animal Said external computer is preferably operatively coupled to the ASP and or means for positioning the ASP and or for positioning parts of said ASP;
b) one at least computers internal to the animal and preferably part of the ASP and or coupled to said ASP. For example, said internal computer is preferably operatively coupled to i) one at least external computers as applicable and or ii) means for positioning the ASP and or iii) for positioning and or controlling parts of said ASP;

Allowance is made for the use of plural ASP's that may communicate with each other and other devices (eg external to the animal), said communication, for example, via one at least known art wired and or wireless means. Said other devices preferably including one at least controlling computers.

Automated Surgical Probes (ASP's) are preferably constructed of materials able to undergo one at least sterilising processes.

It is preferably that motion of an ASP may be controlled/facilitated by external mechanisms, non-limiting examples preferably may include one at least of:
a) an external framework (eg. fixed to the animal and or supporting infrastructure able to position/reposition the ASP within said tissue/cavity as required. Said framework is preferably under computer control and said computer control preferably may be manually overridden in part at least;
b) cable/rod/linkage coupling the ASP to the environment external to the animal.

It is preferable that motion of an ASP may in part at least be independent of external mechanisms and said independent motion is preferably facilitated by one at least motion/positioning/propulsion devices/methods attached to and/or part of said ASP.
Known art motors, actuators and other motion devices, including micro and nano: motors, actuators and motion devices, are readily adaptable for said motion. On the smaller scale these are amenable to fabrication on an integrated circuit type scale. Motion preferably may be facilitated by piezoelectric device and shape metal alloys (eg nano-muscles). Known art micro-robotic devices have ‘legs’ that can walk. It is preferable that a bio-electronic hybrid motor may be used for motion—for example muscles tissue coupled to a semiconductor device that provides electronic stimulation/control—muscle tissue preferably operable from glucose/oxygen in surrounding tissue and electronics preferably powered by known art in-vivo source (eg glucose, vibration). For ASP's at the micro and nano scale, they preferably may be insert-able into one or more cell types of the animal (or a vector) and hitch a rid (for example, embedded in neutrophils may deliver to the site of infection).

Non-limiting examples of enabling an ASP to move its location within the animal preferably may include one at least of:
a) Propulsion through a fluid, that preferably may be natural body fluid and or a fluid synthesised in part at least in vivo (eg by an ASP) and or a fluid provided from an external source, non-limiting examples of said propulsion preferably include one at least of:
   Jet Propulsion (eg squid like),
   Propeller (eg driven by nano motor or bio-electronic hybrid),
   Flagellate (eg driven by nano motor or bio-electronic hybrid),
b) Worm-like motion, eg alternate compression and extension along the axis of travel.
   For example, the front of the ASP attaches to adjacent tissue and pulls the trailing parts towards it and subsequently fixes the back of the ASP to tissue, freeing the front and extending the ASP to push the front forward.
c) Moving Loop Motion—eg Bulldozer track like motion.
d) Mechanical Legs.
e) Building a structure in tissue using the ASP (eg 3D printing) and advancing the ASP from this structure as it is fabricated. It is preferable that part at least of previously fabricated structures may be removed and or modified.

The invention allows that movement may be facilitated by clamping part of the motion apparatus (eg front of tread, tip of leg, front or back of ASP to adjacent tissue, for example to facilitate ‘push off’ or ‘pulling’. Said clamping is preferably reversible. Non-limiting examples of said clamping preferably may include:
Suction to tissue
Wedging—eg the tip of a leg may be placed in a tissue crevice/fold and reversibly inflated to hold it in position.
welding/gluing eg lay down polymer that is set by Type A laser and released by Type B laser. Polymer preferably may be biodegradable. Preferably may attach to tissue and or synthesised (eg plastic) substrate.

It is preferable that motion may be through body fluids and or solid tissue and or natural cavities/lumen and or fabricated cavities/lumen. It is preferable that motion may be facilitated by tissue disruption and or disruption of artificial structures.

It is preferable that the ASP may obtain power from one at least of the following non-limiting examples:
external power delivered by cable
external light source (eg laser) used to power photovoltaic cell in ASP
battery coupled to ASP
power generated from biological milieu (eg glucose in body fluids) or in fluids provided from an external source (eg delivered to a natural or synthetic cavity).
Generated using vibration powered electrical generator located inside an ASP and or coupled to an ASP. These methods are known to the art to generate power for wearable electronic devices and may be adapted for in-vivo use.

It is preferable that part at least of the ASP (eg the tip) may be adjusted horizontally and or vertically and or rotationally with respect to another part of the ASP

The positioning of an ASP within the animal preferably may be facilitated by one at least of:
I) external determination: for example, one at least of:
   a) direct imaging (eg digital imaging)—for example in the case of eye surgery (eg lens replacement surgery) directly via the cornea/anterior chamber;
   b) CT scanning and or MRI (for non-magnetic apparatus);
   c) Known art ocular imaging system;
   d) triangulation from a radio/microwave source coupled directly and or indirectly to the ASP.
II) internal determination: for example direct imaging (eg digital imaging and or ultrasound imaging) by the ASP or another device(s) inserted in-vivo. Said internal determination preferably may include one at least of:
   a) terrain recognition—for example, able to recognise one at least of:
      i) normal anatomical structures;
      ii) abnormal anatomical structures;
      iii) synthetic structures (eg plastic/metal parts constructed in vivo, eg by in vivo 3d printing).
   b) barrier recognition—for example a barrier previously inserted/constructed in vivo, for example, one at least of:
      i) a dye inserted into the space between the lens and lens capsule of the eye may fluoresce when hit by a particular wavelength(s) and detection of said fluorescence may indicate to the system to stop further laser activity beyond this point/region;
      ii) a grouping of nano-electronic device and or nano-motors (that preferably may themselves be able to provide active and or passive signalling (eg R/F), for example detecting particular EMR frequency/intensity, detecting pressure, detecting temperature, detecting vibration;
      iii) 3d printed—eg using biodegradable plastic.
   c) imaging at a cellular/sub-cellular level.
   d) information provided by one at least sensors (eg temperature, pressure, ph, pO2).

It is preferable that an ASP may include tissue/fluid pH measuring capability, (for example to dynamically measure and feedback information on pH changes during one at least of the processes described for an ASP).

It is preferable that an ASP may include pO2 and or CO2 measuring capability, (for example to dynamically measure and feedback information on gas concentration changes during one at least of the processes described for an ASP).

It is preferable that an ASP may include temperature and or pressure measuring capability, (for example to dynamically measure and feedback information on temperature and or pressure changes during one at least of the processes described for an ASP).

It is preferable that an ASP may include a facility for measuring one or more biochemical parameters, haematology, chemicals (eg using integrated micro/nano laboratory).

It is preferable that an ASP may include a facility for nucleic acid sequencing and or synthesis and or editing.

It is preferable that an ASP may include cell recognition and or sorting capability. Said recognition/sorting preferably may be facilitated by one at least cell markers (eg fluorescent dye, antibodies) and or direct imaging of cell/cell contents.

It is preferable that an ASP may include 3D printing capability, for example: plastic, metal, biological material (eg supporting tissue, cells).

### PREFERRED EMBODIMENTS

### Photonic Energy Device

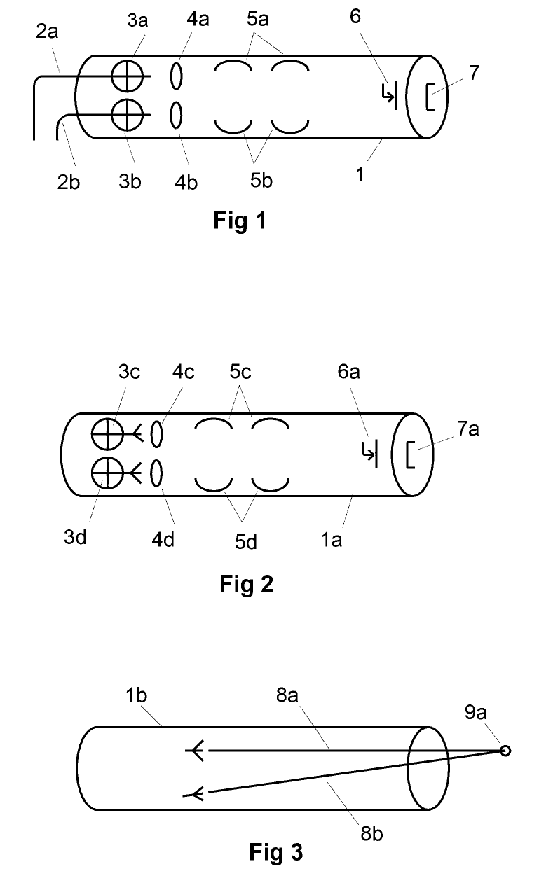

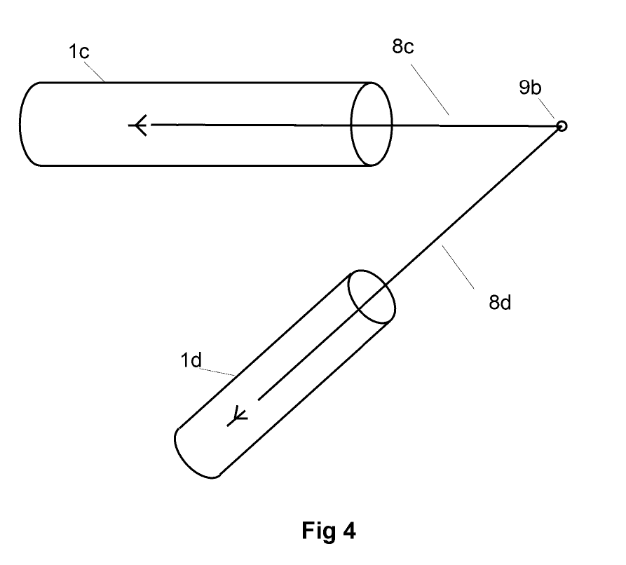

A Photonic Energy Device (PED) to facilitate in-vivo surgery is described with reference to FIG. 1 of the drawings. This preferably may comprise and or be included in an apparatus (eg. a probe or similar surgical instrument) for in-vivo processes that may include one at least of: tissue disruption, targeted cellular destruction (for example, identifying a previously marked (eg by fluorescent techniques) cell or cell group, eg neoplastic cell). Allowance is made that part at least of the photonic energy device may be incorporated into in-vivo surgical instruments, preferably including one at least ASP's disclosed in this specification. The PED permits a plurality of laser sources to be combined into a preferably variable strength beam that may be adjusted by varying the the phases of each laser beam relatively to each other. Completely out of phase minimises total output, whereas completely in phase maximises output. The phase relationship preferably may be adjusted (preferably computer controlled) in response to feed back of effects of the combined beam (eg a) digital imaging of destruction rate, b) temperature/pressure increase, c) intensity of light from laser responsive fluorescing material). PED 1 of FIG. 1 shows fibre-optic conduits 2a and 2b each bringing laser light from a source (eg external Femto or YAG lasers). The position of the outputs from 2a and 2b may be positioned relatively to one another by micro-motors 3a and 3b (thereby altering the phase relationship). Focusing and or phase relationship preferably may be further adjusted by positionable lenses 4a and 4b and positional mirrors 5a and 5b. It will be appreciated that MEMS technology permits large numbers of motors, lens and mirrors to be fabricated in tiny packages. The combined output may be directed using direction mechanism 6 (eg positionable lens). A feed back system 7 is shown, to facilitate determination of output results (eg digital imaging, light level detection, temperature, pressure, detection of various substances resulting from tissue destruction. PED 1a of FIG. 2 shows a similar arrangement however semiconductor laser diodes with adjustment motors are shown at 3c and 3d. Another arrangement is shown in PED 1b of FIG. 3 wherein at least two laser beams 8a and 8b converge outside the device at point 9a. These may be individually adjusted to ensure coherence between the two beams and by varying the angles of the emitted beams they may be made to focus over a large x,y z co-ordinate range, allowing accurate cutting, welding, printing etc in 3 dimensions. In FIG. 4, 1c and 1d represent two distinct PED that may be separately arranged to intersect respective beams 8c and 8d at 9b. It is preferable that vibration generated electricity (preferably augmented) may be used to power part at least of an PED. It will be appreciated that the PED disclosure may have more general application in more macroscopic applications (eg combining lasers for cutting, holography, etc). Accurate tracking and feedback may enhance the power of lasers at considerable distance, including on moving targets.

### Cataract Surgery Probe

A method, system and apparatus for computer controlled or facilitated replacement of a lens in the eye (for example, as a response to cataract formation). Whilst the present disclosure is applicable to a highly automated process, it will be appreciated that parts of the disclosure preferably have application in facilitating eye surgery with varying degrees of manual intervention. Furthermore, processes described for the eye preferably may be adapted/expanded for other in vivo surgery. For example: intervention within living animals that whilst not restricted to, has particular application to humans and other mammals. It is preferable that the cataract probe disclosed in this preferred embodiment may also include one at least functions disclosed for an ASP in this specification.

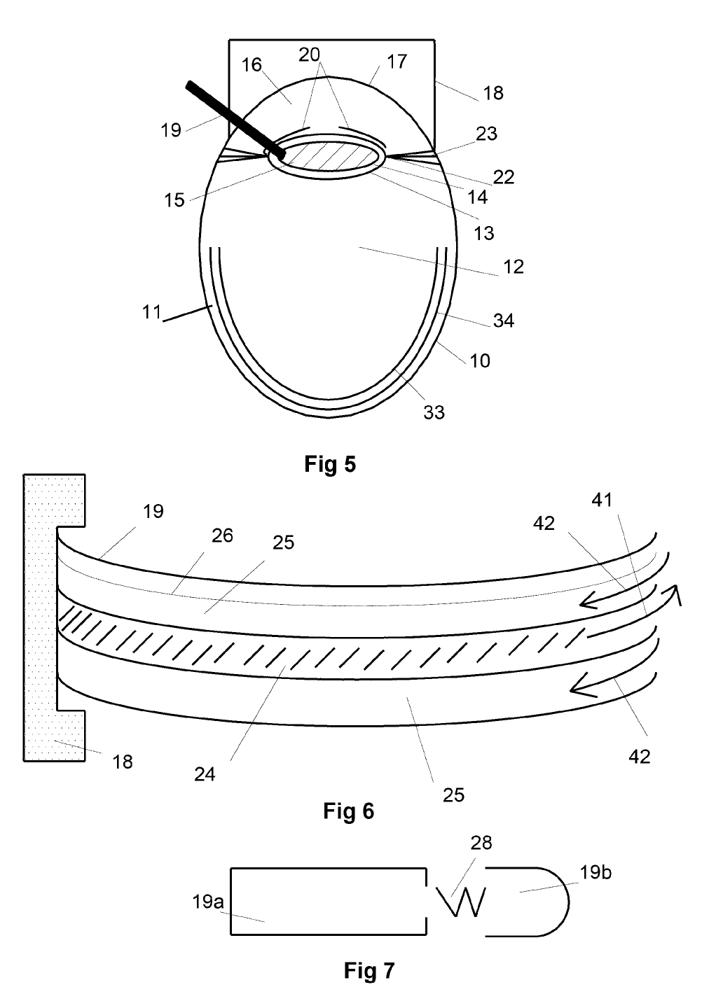

FIG. 5 shows a cross section 10 through an eye, with the cornea 17 forming the front of the eye and also the front of the anterior chamber 16. The iris/pupil 20 cover the crystalline lens 15, that is located behind the iris. The lens 15 is enclosed in lens capsule 13. A space (or potential space) 14 is between the lens 15 and lens capsule 13. It is preferable that the automated process includes the infusion of a fluid (eg elastoviscous) substance into said space 14. Said infused fluid preferably may include one at least of the example ‘flowable protective barriers’ disclosed earlier in this specification. The infused fluid preferably also includes a barrier to indicate (eg material capable of fluorescing in response to laser light of particular frequency(s)) to facilitate determination of when a laser may be encroaching on protected territory. It is preferably a barrier to indicate may be used instead of or together with a barrier to protect/block. The lens is supported by zonules 22 and the ciliary body 23 that also provides the muscle system to facilitate accommodation of the lens. The back of the eye includes the outer sclera 11, the choroid 34 and the retina 33. The vitreous 12 fills the bulk of the region between lens/capsule and the retina. The probe 19 is coupled to external framework 18 (to facilitate movement of the probe within the eye) said framework preferably coupled to an ocular imaging system. The probe 19 preferably may be coupled to an external laser (eg femto or YAG) and or a laser (eg PED) inside the animal, eg within said probe.

In one embodiment the probe 19 is one piece and preferably curved to facilitate movement within the capsule 13. It is designed to remove the material within the crystalline lens of an eye leaving an intact continuous lens capsule with the exception of a small entry wound in the lens capsule.

A curved probe 19 is shown in FIG. 6 with an external casing housing 25 a lower pressure system to aspirate fluid 42 in a direction opposite to the water jet 41. The internal wall of the outer casing 25 is preferably lined with a fibre optic system 26 to enable transmission of laser light energy. Within the probe is a secondary inner tube 24, which houses a smaller diameter high pressure stream that functions as a water jet 41. The flows in both the high pressure water jet, and lower pressure aspiration system is computer controlled and the flow rates in each system are independently controlled. The probe is preferably mounted on external frame 18, which attaches it to a linked ocular imaging system, and optional external femto laser system. This apparatus is in turn coupled to the eye. The horizontal, vertical, and rotary movements of the probe are computer controlled. The function of this probe is to facilitate an automated removal of the material from a human lens capsule. An alternative embodiment in FIG. 7 shows a proximal part of the probe 19a and a distal part 19b that preferably may be extended/retracted/rotated/tilted at 28 independently of movement of proximal part 19a.

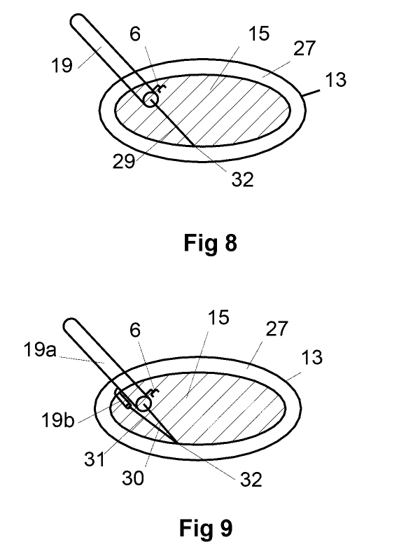

FIG. 8 of the drawings shows the capsule of a lens 13, a lens 15 and the region between the capsule and lens filled with fluid 27. Said fluid preferably provides both an impediment to the passage of a particular wavelength(s) of infrared (for example) from a laser, and emits light when struck by a laser. Both the blocking and signaling characteristics preferably may be by the one agent (eg plastic microbeads may be both blocking and made of a fluorescing plastic). It is also preferable that the laser that requires blocking may also trigger the signal barrier or a separate laser may be used for each of the two functions. ASP 19 includes a laser providing laser beam 29 and a digital imaging device 6. In this example the single beam 29 both disrupts lens material at location 32 where it is adjacent to fluid 27, and also causes the region at 32 to illuminate because of proximity of the beam 29 to fluid 27. The beam 29 may be from one laser with a dual effect, or may be two beams from two lasers (one disruptive and the other for signalling) that are focused together and emerge as one. FIG. 9 is similar however it provides for two laser beam outputs—the first beam 30 from 19a for disruption and the second beam 31 from 19b for signaling. The second beam is directed at the region 32a being disrupted by beam 30 such that illumination at 32a is consistent with concurrent disruptive activity at this point.

The material within a human ocular lens consists of a central harder “nucleus”, and a softer outer cortex. The nucleus has to be removed by its disassembly by fragmentation or emulsification. The softer material of the cortex can be removed by aspiration. The Fibre Optic light system is designed to deliver laser light energy to fragment the lens nuclear material. The Water Jet 41 is designed to help break up the nucleus material, to provide irrigating fluid, and to cleave the soft cortical material free from the lens capsule. The water jet can be used to manipulate the position of lens material. The Aspiration system is designed the remove gas, heat, and material created by the fragmentation process. By increasing the flow rate in the aspiration system relative to the water jet, the water jet can be opposed to the hard nuclear material.

The respective flow rates of the inflow and outflow channels of the probe can be varied independently of each other, to enable the manipulation of tissue fragments, aspiration of material, and can function as a water jet to erode tissues. The water jet can be used to cleave soft lens cortical material away from the capsule membrane, for later aspiration.

The inflow channel preferably may be used to introduce the protective (eg infrared opaque viscous fluid) and or other fluid (eg defining barrier) between the soft lens matter and the lens capsule. As lens material is cut/removed is preferable that additional protective and or defining material may be introduced into the expanding spaces. As appropriate it is preferable that introduced fluid is removed (eg by aspiration). It further preferable that any remaining ‘fluid’ is biodegradable.
The Delivery Conduit preferably has fluid at higher pressure than fluid in the Removal Conduit.
The Removal Conduit is preferably optimised for use as an aspiration channel.
It is preferable that the Delivery and Removal Conduits have independently controllable flow rates that preferably may each be automatically or semi-automatically adjusted.
The Delivery Conduit is preferably narrower than the Removal Conduit.
The Delivery Conduit preferably may comprise a plurality distinct conduits. It is preferably that different functions may be provided for by a a particular member of said plurality.
The Delivery Conduit preferably permits fluid to be delivered into the lens as a jet (at or otherwise focused fluid stream). Said focus is preferably variable
Fluid from the delivery conduit is preferably able to disrput lens material. The fluid output pressure/force is preferably variable. It may preferably be pulsed. Said pulse rate is preferably variable.
The position of the Delivery Conduit within the probe is preferably central compared to the Removal Conduit. It is preferable that the Removal Conduit surrounds the Delivery Conduit in part at least.
The Delivery Conduit is preferably optimised to facilitate/enable the delivered fluid to cut and or otherwise destroy lens tissue, said cutting/destruction part of a controlled process.
The Delivery Conduit and or apparatus coupled to the probe preferably allows the flow rate and or flow pressure and or flow direction as it exits the probe and or the frequency of any pulsation of pressure, to be machine controlled (said machine control preferably including, in part at least, computer control). Said flow characteristics are preferably independent of those that may apply to fluid in the Return Conduit.
The probe preferably includes and or is coupled to a device(s) for measuring temperature and or pressure in one at least parts of the lens. Said part of the eye preferably includes in the vicinity of the probe tip and or where active lens destruction is occurring. The preferred device for measuring temperature is a digital means.
The probe preferably includes a conduit suitable for inserting a new lens inside the lens capsule.
Computer controlled mechanism (eg motor) preferably able to operate tip movement (eg horizontally and/or vertically and/or in rotation and or extension and or retraction).
Said automation preferably includes computer control that may include one at least of:
   remote computing (preferably coupled by a WAN eg internet);
   local computing local to the patient and preferably coupled to the probe by wireless and or wired communications;
   computing integrated into the probe.
Said Automation preferably includes an external framework (preferably including moveable electromechanical parts able to facilitate positioning of the probe and or imaging system and or one or more external lasers and or one or more internal lasers (eg directed through the fibre-optic conduit).
Said automation preferably includes the use of digital imaging means/device/software that in part at least may be external to the eye and/or internal to the eye (eg part of the probe).
The fibre-optic conduit preferably forms part of a system that in conjunction with other parts of the probe and/or other supporting apparatus (non-limiting examples: computer, laser light source(s); electro-mechanical devices for controlling movement and or positioning of the probe; imaging systems for detecting eye movement, position of the probe relative to the eye and or within the eye; apparatus to determine the progress of lens destruction) controls and or provides laser light such that one at least of the following preferably apply:
   a) laser light is suitable for use in the cutting and or otherwise destruction of part at least of said lens material.
   b) laser light is delivered using a method that provides for the controlled cutting and or otherwise destruction of part at least of said lens material, and in particular under the control (in part at least) of apparatus and/or control system that preferably eliminates and at least minimises damage or the probability of damage to the lens capsule.
   c) laser light that can be focused and or otherwise controlled/modified such that the region of cutting and or otherwise destruction of lens material relative to its exit from the probe (eg: the probe tip) may be constrained and or otherwise controlled.
   d) laser light of different frequencies or other characteristics may be delivered separately and or concurrently. For example, light of a particular characteristic may be used for cutting and or vaporising lens material, another may be used to activate a material to make it impervious to laser light (eg to protect part of the lens and or surrounding tissue from laser light) and yet another may be used to deactivate a protective fluid (eg alter a dye from blue to transparent).

The cataract embodiment seeks to provide for a number of methodologies for automating the removal of lens material. Advantages may include lower cost procedures, less damage to the eye, increased versatility on what may be achieved within the lens capsule.

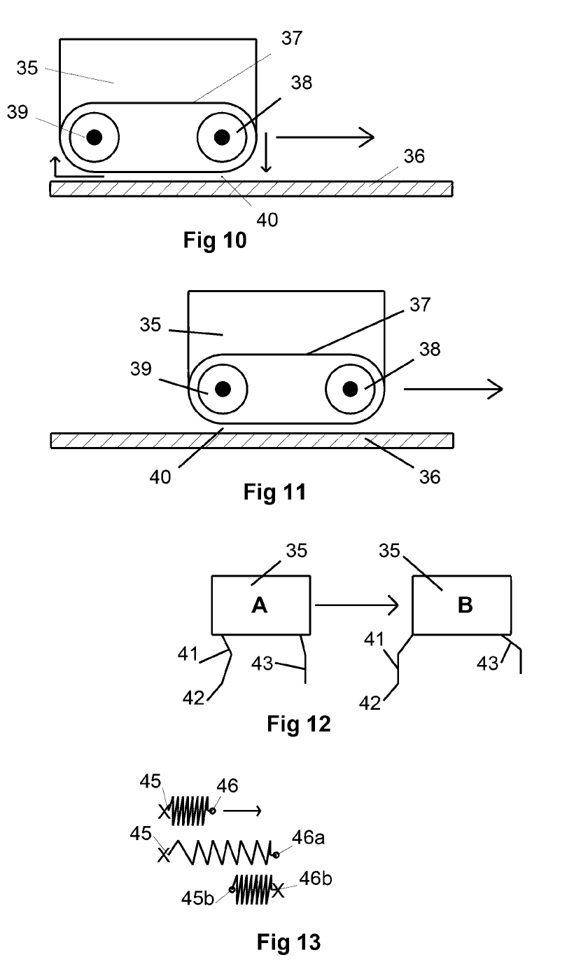

Several methods for mobilising an ASP are now described with reference to FIGS. 10, 11,12 and 13 of the drawings. A structure (eg tissue or artificial) 36 for ASP 35 to move along is shown. ASP 35 in FIGS. 10 and 11 are the same ASP in different positions along the identical section of structure 36. The ASP includes a looped belt that moves around rear wheel (or similar) 39 and front wheel (or similar) 38. The ASP temporarily fixes a location on the belt 37 with the structure 36. Rotation of the wheels 38 and 39 move the ASP forward, however the the point of connection between belt 37 and structure 36 is unchanged. The next step is to break the bond at 40 and move it forward along the track to enable continued forward motion. Reversing the steps permits reverse motion. ASP 35 of FIG. 12 uses mechanical legs for movement. In position A, ASP 35 has the rear leg 41 fixed to adjacent structure at location 42. ASP 35 then moves forward to position B by extension/hyperextension of rear leg 41 that remains fixed at point 42. For subsequent (not shown) forward motion, front leg fixes (eg laser melting of plastic) to the structure and rear leg 41 is unfixed (eg laser cutting). Extrusion of a polymer through a channel inside said legs that exits at the end of the leg preferably facilitates fixing of the leg to the structure. A compression/extension method is depicted with 45 and 46 of FIG. 13. The rear of the mechanism 45 is initially fixed as shown by the X with the mechanism compressed and the front 46 unattached. In the next step extension occurs moving the front to a forward position 46a. The rear is still fixed. The next step is to fix the front (same location as 46a however now fixed and identified as 46b with the rear freed and pulled forward to the position 45b.

### Example ASP

An ASP is preferably a small, intelligent and adaptable instrument, that under computer control (that is preferably within said ASP in part at least) with feedback from one or more sensors may intervene (eg surgery) inside the animal with a greater or lesser amount of autonomy. It preferably includes digital imaging and preferably is able to transfer said imagery (and or other information) externally (eg to a doctor). It is envisaged that as the software is refined, the ASP may be capable of performing intricate surgery and or other in-vivo processes unaided. It is envisaged that ASP's may be versatile mobile medical instrumentation responsive to a variety of downloadable Apps for directing a variety of surgical and medical interventions.

Ongoing refinements in micro electronics, micropower generation and MEMS is expected to reflect in the evolution of smaller and more versatile ASP's.

It is preferable that an ASP(s) may be left inside the animal for extended periods.
Standard surgery performed under anaesthesia, relies on opening the patient and getting them closed again as quickly as possible.
The ASP allows for surgery to be performed over a continuous period a bit at a time.
   For example an ASP embedded in the front of the vitreous may progressively tackle a lens problem, a portion at a time. An assessment of the results of a first stage may be made before tackling another. This may also be the case for replacing vitreous and or embedding microelectronics into the retina to improve vision as a non-limiting example
   ‘Of course, it may be a little bit annoying to see the ASP ‘walking around’ in your eye, but hey! At least while you have a surgeon in your eye, you can keep an eye on your surgeon.’

It is preferable that the ASP may move parts of itself relative to each other and also move within the animal. Said movement preferably may be facilitated by external infrastructure and or by motion apparatus attached to the ASP (eg mechanical legs, belt loop etc). The ASP may also fix part of itself to surrounding structures that may be biological and or synthetic (eg previously constructed by the ASP). The fixture may be transient (eg holding a mechanical leg in place while it pushes the ASP forward) or more robust (eg fixing the ASP in place while it operates)—this may be particularly applicable in locations where it may be swept away (eg in a blood vessels, the bladder or the gut.

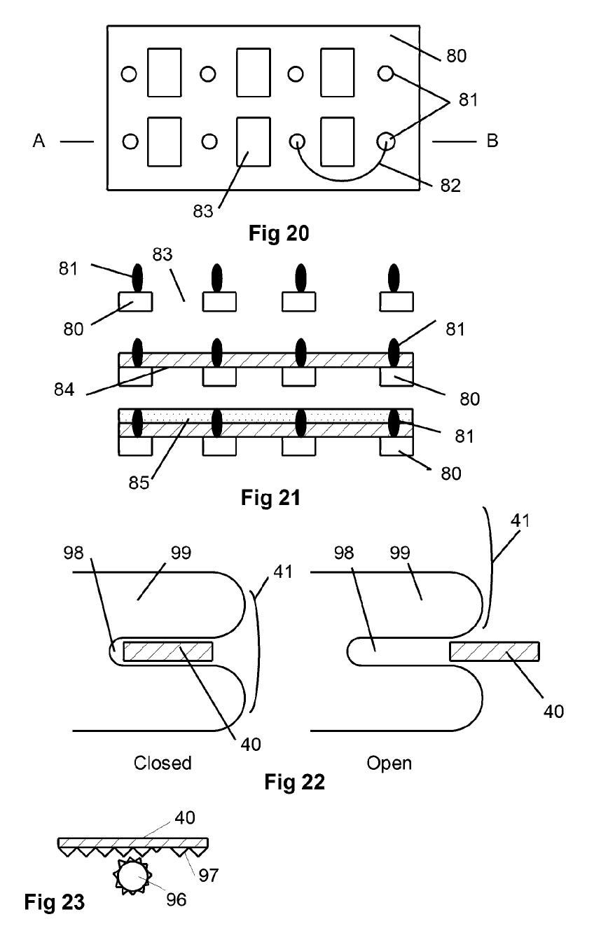

In one embodiment, the ASP, while preferably still subject to external control, is standalone and includes the necessary tools and resouces (and or is capable of manufacturing those resources from available substances, eg: in the animal tissue/fluids, stored items or recycled items). The preferred power units comprise one at least of: battery, vibration electricity generator, and or energy derived from available resources in the tissue (eg glucose). The latter option may be particulalry applicable for an ASP with hybrid biological (eg muscle) and synthetic structures:
An example bioelectronic hybrid is described with reference to FIGS. 20 and 21: wherein a thin polymer sheet 80, with fenestrations 83, electrodes 81 (preferably printed conductive polymers), and electrical conductors 82 (preferably printed conductive polymers) is shown viewed from above in FIG. 20. It preferably may have other electronic devices, for example: a) polymer printing of diode, capacitors, semiconductors or b) silicon based electronics. Step 1 of FIG. 21 shows a cross section through AB. Step 2 shows a fibrous sheath 84 that has been grown on the substrate 80. Step 3 depicts the top layer of muscles cells 85 grown onto the supporting fibrous sheath 84. Blood vessels and other supporting structures are not depicted. The biological tissue preferable receives nutrient via blood vessel, artifical conduits and illusion from surrounding tissue. The muscle 85 preferably may be stimulated by the printed electrodes 81.

It is preferable that the ASP may have access to another ASP and or a docking station with internal and external portals for replenishment, waste removal etc. In another preferred embodiment, the ASP is physically coupled by one at least by cables (eg wire, fibre) and or conduits to an internal docking station and or directly to external resources.

A preferred ASP tool is a Suturing Tool for joining together a plurality of pieces of tissue

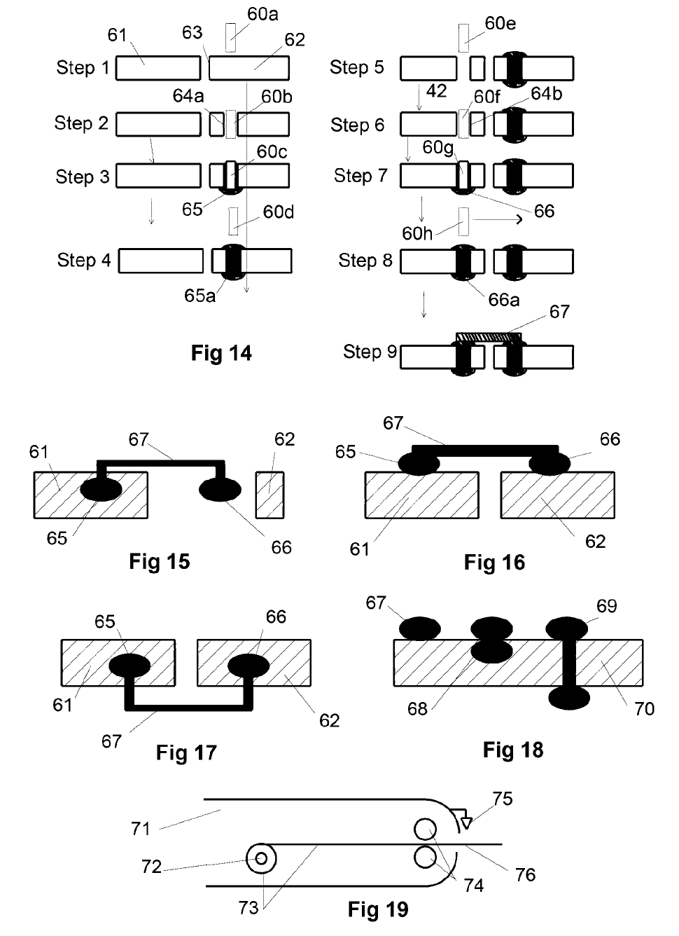

An example method step and apparatus for automated joining of tissue (eg suturing) by an ASP is described with reference to FIG. 14 of the drawings. While a single probe 60 is described, it is preferable that the number of probes coupled to an ASP is not limited.
Step 1 shows two pieces of tissue 61 and 62 that have been opposed (eg by an ASP's micro forceps) with an intervening gap. A probe 60 (identified as 60a, 60b etc in successive steps) is coupled to an apparatus that enables it to be positioned and moved up and down. It may includes a conduit for extruding a polymer out its tip. Examples of said polymer may be liquid, solid (eg powder), or a thread. Examples methods of setting the polymer may include: setting on exposure to light (eg UN or visible) or melting (eg laser, U/S welding, direct heat) or curing as a glue or a two part polymer solution. The probe 60 preferably includes one at least of: laser output, light output, heat source and or US at or the vicinity of the tip for setting the polymer, source and delivery of setting catalyst. A tip related energy source preferably may be used for other ASP functions (eg deleting unwanted structures, cautery). Step 1 shows the tissue pieces opposed and the probe 60a above tissue 62. In Step 2 the probe 60b has punched/pushed a hole 64a in tissue 62. In Step 3 the probe 60c extrudes and sets a securing pad of polymer 65 on the underside of tissue 62. In Step 4 the probe 60d progressively withdraws along opening 64a, extruding and setting polymer as it withdraws, preferably leaving a pad on the top surface of tissue 62. In Step 5 the probe 60e is then positioned over tissue piece 61, in Step 6 the probe 60f has punched/pushed a hole 64b in tissue 61. In Step 7 the probe 60g extrudes and sets a securing pad 66 of polymer on the underside of tissue 60. In Step 8 the probe 60h progressively withdraws along opening 64b, extruding and setting polymer as it withdraws, preferably leaving a pad on the top surface of tissue 61. In Step 9 the probe moves across both pieces of tissue extruding a setting a polymer bridge 67 that anchors tissue 60 and 61 together.

Some alternative suturing examples are described with reference to: FIG. 15, wherein the securing pads 65 and 66 are embedded in there respective tissue pieces 61 and 62—the probe extrudes and sets material into the tissue instead of on the other side. Pads 65 and 66 are joined by bridge 67. FIG. 16 depicts fixing pads 65 and 66 adherent to the surface of the respective tissue pieces 61 and 62.

FIG. 17 is similar to FIG. 15 however the fixing pads 65 and 66 are inserted from the underside of tissue pieces 61 and 62 respectively with bridge 67. An example application for the FIG. 17 arrangement preferably includes joining skin together, with the top of the tissue 61 and 62 correlating with surface of the skin. ASP facilitate suturing from outside in. Useful for cosmetic surgery.

It will be appreciated that the methods depicted with reference to FIGS. 14, 15, 16 and 17 may also have other applications.
For example, this type of fixing may be used to assist an ASB to move (eg alternate fixing and releasing (eg: cutting, burning) of the end of a mechanical leg), and or to attach and hold an ASB in place and or to provide a base for construction of synthetic structures.

FIG. 18 depicts a contiguous piece of tissue 70 with several example fixing pads: 67 (adhered to the surface of tissue 70), 68 (embedded in the tissue 70) and 69 (attached through the tissue 70 to the bottom surface). Examples of the tissue 70 may include soft tissue, bone, wall of a blood vessel or bronchus, wall of the GIT. Structure 70 preferably may also be non-biological, for example a synthetic structure deposited by the ASP or a synthetic structure provided from an external location (which may have been imported in pieces and fused together internally by the ASP).

It will be further appreciated that the methods depicted in FIGS. 14 through 18 provides a method and apparatus for building synthetic and or biological structure within an animal. The methodology is fundamentally 3d printing of structures within a living animal that includes: the depositing (sequentially or in parallel) of building blocks and the fixing of them in place by fusing to adjacent structure. Examples of said building blocks may include: a) droplet of a fluid (eg polymer, resin), b) powder (eg plastic microbeads, metal powder) c) fibres (eg polymer, carbon fibres, metal fibres), d) ink for printing electronic device and conductors, e) silicon semiconductor (eg delivered on tape that may be placed using a similar tool to that of FIG. 19 and used in conjunction with a forceps/cuttingtool), components imported from outside the animal (eg the bioelectronic hybrid of FIGS. 20 and 21, that in preferably may alternatively be constructed in vivo), f) self assembling microelectronics, g) carbon nano tubes that preferably self assemble, h) biological connective tissue (eg collagen, elastin, fibrin), i) cells (eg stem cells, cultured cells) and j) proteins (eg synthesised by ASP), k) chemicals, l) cytotoxics, m) enzymes.

An example apparatus for delivering fibre from an ASP may include a spool of fibre that may be fed onto a worksite. FIG. 19 depicts a probe 71, that is preferably a retractable extension from an ASP, a spool of fibre 73 on an axle 72 that may be moved out (or in) by rollers 74 through opening 76. The fibre may be cut by shear 75. Fibre cutting preferably may use other means, for example, another ASP tool with shears that may have other uses.

Examples of methods for joining adjacent building blocks may include: exposure to light, laser, heat, ultrasound, multipart polymers, curing, protein linking.

The concurrent availability of laser, ultrasound, fluid jets and a variety of cutting and polishing tools further coupled to ASP's further facilitates in vivo 3d printing and finishing, polishing and removal of unwanted parts. The Photonic Energy device that provides accurate focussing in all 3 cartesian co-ordinates may also assist.

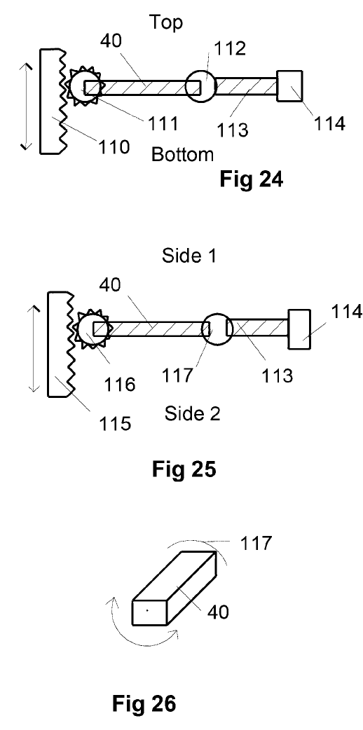

FIG. 22 depicts an embodiment for a tool storage pod for tools coupled to or part of an ASP. The pod 99 includes a storage bay 98 and shows a generic tool 40. When not in use (closed) the tool 40 is retracted and cover 41 closed. When in use (open) the cover is opened and the tool projected out. Examples configuration of the pod include protruding from the ASP or buried within. It is preferable that the pod may store multiple tools and use a micromechanism to switch them as required. FIG. 23 shows an example method of moving tool 40 in and out by using a row of linear teeth 97 along the bottom of tool 40 that meshes with toothed cog 96. Movement of the tool in and out may also facilitate operation of the tool (eg a knife on the end may be able to cut). FIG. 24 is a view of the extended tool 40 from the side. It may move up and down using the teeth on 110 together with the toothed cog 111. The distal end 113 with the active part of the tool 114 is able to move up and down in the arc shown around pivot 112. FIG. 25 is functionally similar to FIG. 24. It shows tool 40 from above. The teeth on 115 and 116 permits linear side to side motion and the pivot 117 permits the distal part 113 to move in an arc sideways. FIG. 26 is an end view showing rotation around the long axis.

Example tools may include: light source, laser, ultrasound probe, hot tip, fluid jet, delivery conduit (eg fibre, liquid polymer, plastic granules, metal granules, nutrients, chemicals, biological material), removal conduit, scalpel, blunt probe, suturing probe, scissors, forceps, intracellular probe, cutting disc, polishing wheel/disc, circular saw, clamp.

### Accommodating Lens

Whilst current ophthalmic intervention on the lens is predominantly to remove a cataract and replace it with an artificial lens, an ASP preferably may have application intervening earlier to solve the problem of lost lens elasticity associated with presbyopia—a problem affecting a much younger age group than cataract formation.

For example: Part only of the lens may be removed and replaced (eg externally supplied component or 3d printed in-situ). The entire lens may be removed and replaced (the replacement preferably may be an entire lens or one provided part at least as components that are joined (eg fused, glued) together in situ and or 3d printed in part at least). The new material is preferably responsive to ciliary muscle activity. The use of nano-technology may have application here. It may be than adaptive lens comprises a mix of biologically compatible material and micro/nano electronic devices that are powered by tissue fluids and or nerve impulse and can be programmed to adapt the lens shape accordingly. It may be that artificial devices are coupled to ciliary muscle and or nerves supplying the same.

It is envisaged that machine controlled sculpting of lens material and or adjacent structures with a fluidjet and or laser light may be able to carve approriate niches to facilitate coupling of any adaptivelens structure to existing anatomical structures.

The claims appended to this document include part of the present invention and are incorporated into this specification by way of reference.

1-78. (canceled)

79. A method of performing a procedure within the capsular structure of the lens of an eye that maintains the integrity of the barrier between lens and anterior chamber during said procedure, wherein said procedure comprises at least one of:
extraction of lens cortex material,
extraction of lens nuclear material,
provision of a replacement lens.

80. The method of claim 79 wherein said procedure includes manual or computer controlled application of electromagnetic radiation to disrupt lens material.

81. The method of claim 80 wherein said electromagnetic radiation includes laser light applied to the lens using one at least of: a) directed from outside the eye through the anterior chamber into the lens, b) emerging from an instrument inserted into the eye.

82. The method of claim 81 wherein said instrument inserted into the eye provides multiple laser sources computer controlled to elicit a combined effect.

83. The method of claim 82 wherein said control includes variation of the phase relationship between two lasers.

84. The method of claim 82 wherein said combined effect provides for precise delivery of laser energy in three dimensions.

85. The method of claim 81 including provision of a barrier inside the lens capsule to protect against unwanted damage from laser energy.

86. The method of claim 79 including provision of an indicating barrier to facilitate computer determined detection of the progress of disruption of lens material.

87. The method of claim 79 further comprising an instrument inserted into the eye, said instrument for acquiring at least one of: digital imagery, ultrasound measurement, pH, temperature, pressure, pO2, cell recognition within the lens capsule,
and
said acquired information is processed in the computer determination of subsequent disruption and or removal of lens content.

88. The method of claim 79 further comprising the use of at least one computer controlled mirror or lens located within the eye.

89. The method of claim 79 further comprising the insertion of an instrument within the lens capsule, said instrument providing computer controlled inflow or outflow of fluids for use in at least one of: disruption of lens content, removal of lens content.

90. An instrument for insertion inside a lens capsule for the computer controlled disruption and or removal of lens material, said instrument including at least one of: computer controlled inflow of fluids, computer controlled extraction of fluids, computer controlled delivery of laser energy, a conduit for insertion of a replacement lens.

91. The instrument of claim 90 further comprising the computer aided recognition of an indicating barrier, said recognition determining subsequent disruption of lens material.

92. The instrument of claim 90 further comprising computer controlled independent adjustment of fluid inflow and outflow to enable the manipulation of tissue fragments, aspiration of material, erosion of tissue.

93. The instrument of claim 90 further comprising a computer controlled mechanism to rotate, extend, retract, move horizontally, move vertically; part of said instrument.

94. The instrument of claim 90 further comprising a computer controlled scalpel, probe, scissors, forceps.

95. The instrument of claim 94 wherein said computer control is a response to automated acquisition of information about the contents of the lens capsule.

96. A barrier for use within the lens capsule of an eye wherein said barrier reduces unwanted damage to said capsule by laser radiation and said radiation facilitates removal of lens material.

97. The barrier of claim 96 wherein said barrier includes a fluid component.

98. A system for the controlled deposition of 3d printable material insitu within a human or other animal.

99. The system of claim 98 wherein said controlled deposition includes the use of laser energy exiting a device inside said animal.

100. The system of claim 98 wherein said controlled deposition includes the use of multiple lasers to provide a combined effect at a particular location.
